(19) United States
(12) Patent Application Publication
(10) Pub. No.: US 2004/0191871 A1
(43) Pub. Date: Sep. 30, 2004

(54) Method for plasmid preparation by conversion of open circular plasmid

(76) Inventor: Edward David Hyman, River Ridge, LA (US)

Correspondence Address: NIXON & VANDERHYE, PC, 1100 N GLEBE ROAD, 8TH FLOOR, ARLINGTON, VA, 22201-4714, US

(21) Appl. No.: 10/396,880

(22) Filed: Mar. 25, 2003

(51) Int. Cl. C12P 19/34; C12N 15/00

(52) U.S. Cl. 435/91.1; 435/320.1

(57) ABSTRACT

In accordance with the invention, there is provided a method for preparing plasmid from host cells which contain the plasmid, comprising the steps: (a) preparing a cleared lysate of the host cells, wherein the cleared lysate comprises unligatable open circular plasmid, wherein the open circular plasmid is not 3′-hydroxyl, 5-phosphate nicked plasmid; (b) incubating the unligatable open circular plasmid with one or more enzymes in the presence of their appropriate nucleotide cofactors, whereby the unligatable open circular plasmid is converted to 3′-hydroxyl, 5′-phosphate nicked plasmid; (c) incubating the 3′-hydroxyl, 5′-phosphate nicked plasmid with DNA ligase in the presence of DNA ligase nucleotide cofactor, whereby 3′-hydroxyl, 5′-phosphate nicked plasmid is converted to relaxed covalently closed circular plasmid; and (d) incubating the relaxed covalently closed circular plasmid with DNA gyrase in the presence of DNA gyrase nucleotide cofactor, whereby relaxed covalently closed circular plasmid is converted to negatively supercoiled plasmid. Preferably, the enzymatic steps (b), (c), and (d) are performed in a single step using an enzyme mixture comprising DNA polymerase, DNA ligase, and DNA gyrase. Preferably, the mixture further comprises a 3′ terminus deblocking enzyme, such as exonuclease III or 3′-phosphatase. Preferably, the mixture further comprises one or more regenerating enzymes and a high energy phosphate donor, whereby the nucleotide by-products of the nucleotide cofactors generated by DNA ligase and DNA gyrase are converted to back to nucleotide cofactor. Preferably, the enzyme mixture further comprises one or more exonucleases, such as ATP dependent exonuclease, whereby linear chromosomal DNA is selectively degraded.

### BACKGROUND OF THE INVENTION

Plasmids are double stranded, circular, extrachromosomal DNA molecules. Plasmids are defined in this invention as such. Plasmids are contained inside host cells. A common host cell is Escherichia coli (E. coli). Many other types of cells are known to carry plasmids. This includes other bacteria, yeast, and higher eukaryotic cells. Plasmids may be man-made, such as cloning vectors carrying foreign DNA inserts. Plasmids may also occur naturally, such as mitochondrial and chloroplast DNA.

Since the invention of cloning circa 1975, the preparation of plasmid has been a routine task in molecular biology research. In the ensuing 25 years to the present time, the art of plasmid preparation has become a highly crowded art. The crowded nature of the art is a reflection of the widespread importance of the procedure in molecular biology. Over 175 articles and numerous patents have been published in the past 25 years describing novel methods for preparing plasmid. The problem of plasmid preparation has attracted enormous commercial interest. Numerous commercial companies sell kits for plasmid preparation (Amersham, Qbiogene, Clonetech, Promega, Biorad, Qiagen). Numerous companies sell proprietary resins for purifying plasmid (Qiagen, Puresyn, Macherey-Nagel). Several companies sell automated instruments for preparing plasmid (Qiagen, MacConnell, Autogen).

In the purification of plasmid from host cells, the final plasmid preparation is a mixture of two main forms of plasmid: open circular and supercoiled. In the supercoiled form, the plasmid has a covalently closed circular form, and the plasmid is negatively supercoiled in the host cell by the action of host enzymes. In the open circular form, one of the strands of the DNA duplex is broken. The single strand break in open circular plasmid results in a relaxed topology.

Open circular plasmid in a plasmid preparation can result from several causes. Open circular plasmid may exist in the host cells immediately prior to lysis. Supercoiled plasmid may unintentionally be converted to open circular plasmid in the preparation of a cleared lysate. Additional plasmid purification steps, such as organic extraction, precipitation, and chromatography, may unintentionally convert supercoiled plasmid to open circular plasmid. This conversion may be caused by several factors. Phosphodiester bonds can be hydrolyzed by thermal hydrolysis, acid hydrolysis, alkaline hydrolysis, free radicals, or heavy metals. Free radicals may damage the ribose sugar or base, resulting in single stranded breaks in the plasmid.

The open circular plasmid may be nicked plasmid or may be gapped plasmid. The 3′ and 5′ terminal ends of the single strand break may be ordinary hydroxyl or phosphate groups. Alternatively, the terminal ends may be functional groups other than hydroxyl or phosphate. For example, free radical damage usually produces single stranded breaks with non-ordinary termini, such as 3′-phosphoglycolate or 5′-aldehyde.

For most plasmid applications, the active plasmid form is the supercoiled form. Open circular plasmid is either inactive or poorly active. Plasmid for human therapy requires a high percentage of supercoiled plasmid and a low percentage of open circular plasmid contamination. Numerous methods are described in the art to achieve this objective.

Saha et al describe a method for purifying supercoiled plasmid from open circular plasmid using agarose gel electrophoresis (Saha, 1989, Analytical Biochemistry, 176, 344-9). Separation is based on differential migration in agarose gel. Supercoiled plasmid is recovered from the ethidium bromide stained gel.

Gorich et al describe a method for purifying supercoiled plasmid from open circular plasmid using polyacrylamide gel electrophoresis (Gorich et al, 1998, Electrophoresis, 19, 1575-6). Separation is based on differential migration in polyacrylamide gel. Supercoiled plasmid is recovered from the gel by electrophoretic elution.

Womble describes a method for purifying supercoiled plasmid using density gradient centrifugation (Womble et al, 1977, J. Bacteriology, 130, 148-53). Plasmid is dissolved in a cesium chloride ethidium bromide solution and centrifuged at high speed. Supercoiled plasmid is separated from open circular plasmid based on differential incorporation of ethidium bromide.

Hyman describes a method for purifying supercoiled plasmid using selective exonuclease digestion (Hyman, 1992, Biotechniques, 13, 550-4). A cell lysate is incubated with a mixture of exonuclease I and exonuclease III. The exonucleases selectively degrade open circular plasmid and chromosomal DNA without degrading supercoiled plasmid, thereby purifying supercoiled plasmid.

Best et al describe a method for purifying supercoiled plasmid using reverse phase chromatography (Best et al, 1981, Analytical Biochemistry, 114, 235-43). The chromatographic resin separates supercoiled from open circular plasmid. Many chromatographic methods are described in the art for purifying supercoiled plasmid from open circular plasmid. This includes reverse phase, anion exchange, size exclusion, membrane, and thiophilic chromatography. Several chromatographic resins are commercially available for separating supercoiled from open circular plasmid (Puresyn, Amersham, Prometic).

All prior art methods for purifying supercoiled plasmid from the open circular plasmid teach separation and removal of the open circular plasmid from the supercoiled plasmid, or teach selective destruction of the open circular form. In the chromatographic, electrophoretic, and ultracentrifugation methods for purifying supercoiled plasmid, the open circular plasmid is separated and discarded. In the enzymatic purification method, open circular plasmid is selectively degraded by an enzyme mixture. One disadvantage of prior art approaches is that the final yield of supercoiled plasmid is reduced, because open circular plasmid is discarded. For example, large scale plasmid preparations may contain 10% to 30% open circular plasmid. Using prior art methods, at least 10% to 30% of the total plasmid will be lost in order to achieve purified supercoiled plasmid.

To the inventor's knowledge, no method exists for purifying supercoiled plasmid which preserves the open circular plasmid.

### OBJECTS OF THE INVENTION

Accordingly, the object and advantage of the invention is to provide a method for preparing supercoiled plasmid, by converting the open circular plasmid into supercoiled plasmid enzymatically, thereby achieving a final plasmid preparation which has a high percentage of supercoiled plasmid.

Further objects and advantages will become apparent from a consideration of the ensuing description.

### DESCRIPTION OF DRAWING

FIG. 1: The method of the invention.

### SUMMARY OF THE INVENTION

In accordance with the invention, there is provided a method for preparing plasmid from host cells which contain the plasmid, comprising the steps: (a) preparing a cleared lysate of the host cells, wherein the cleared lysate comprises unligatable open circular plasmid, wherein the unligatable open circular plasmid is not 3′-hydroxyl, 5-phosphate nicked plasmid; (b) incubating the unligatable open circular plasmid with one or more enzymes in the presence of their appropriate nucleotide cofactors, whereby the unligatable open circular plasmid is converted to 3′-hydroxyl, 5′-phosphate nicked plasmid; (c) incubating the 3′-hydroxyl, 5′-phosphate nicked plasmid with DNA ligase in the presence of DNA ligase nucleotide cofactor, whereby 3′-hydroxyl, 5′-phosphate nicked plasmid is converted to relaxed covalently closed circular plasmid; and (d) incubating the relaxed covalently closed circular plasmid with DNA gyrase in the presence of DNA gyrase nucleotide cofactor, whereby relaxed covalently closed circular plasmid is converted to negatively supercoiled plasmid.

Preferably, the enzymatic steps (b), (c), and (d) are performed in a single step using an enzyme mixture comprising DNA polymerase, DNA ligase, and DNA gyrase. Preferably, the mixture further comprises a 3′ deblocking enzyme, such as exonuclease III or 3′-phosphatase. Preferably, the mixture further comprises one or more regenerating enzymes and a high energy phosphate donor, whereby the nucleotide by-products of the nucleotide cofactors generated by DNA ligase and DNA gyrase are converted to back to nucleotide cofactor. Preferably, the enzyme mixture further comprises one or more exonucleases, such as ATP dependent exonuclease, whereby linear chromosomal DNA is degraded, substantially without degrading open circular or supercoiled plasmid.

### DETAILED DESCRIPTION OF THE INVENTION

Four separate arts have been well established in the literature. (1) In the art of DNA repair, the enzymatic repair of single stranded breaks in double stranded DNA is well established. In 1972, Laipis used DNA polymerase I and DNA ligase to repair single stranded breaks (Laipis et al, 1972, Proc. Natl. Acad. Sci. USA, 69, 3211-4). In 1976, Mitzel-Landbeck used exonuclease III, DNA polymerase I, and DNA ligase to repair single stranded breaks (Mitzel-Landbeck et al, 1976, Biochim Biophys Acta, 432, 145-53). (2) In the art of DNA replication, the conversion of covalently closed circular plasmid to supercoiled plasmid is known to be accomplished by DNA gyrase, discovered in 1976 (Gellert, 1976, Proc. Natl. Acad. Sci. USA, 73, 3872-6). (3) In a study of DNA replication, Shlomai converted open circular plasmid, generated from single stranded circular DNA, to double stranded supercoiled plasmid using an enzyme mixture comprising DNA polymerase I, DNA ligase, and DNA gyrase (Shlomai et al, 1981, J. Biol. Chem., 256, 5233-8). (4) In the art of plasmid preparation for 25 years, a high percentage of supercoiled plasmid and a low percentage of open circular plasmid is known to be desirable. To accomplish this task, numerous methods for purifying supercoiled plasmid have been devised.

The invention described herein is a synthesis of these four arts. The result of this synthesis is an improved method for plasmid preparation.

In the invention, open circular plasmid is enzymatically converted to supercoiled plasmid. This is accomplished by incubating the open circular plasmid with a series of enzymes, either sequentially or preferably simultaneously with an enzyme mixture. The result of this enzymatic method is a plasmid preparation with a higher percentage of supercoiled plasmid and lower percentage of open circular plasmid. The invention operates in a fundamentally different manner from all prior art teaching. In the invention, open circular plasmid is not separated and is not degraded from supercoiled plasmid.

### Preparing the Cleared Lysate

The enzymatic steps of the invention are performed after obtaining a cleared lysate of the host cells containing the plasmid. A cleared lysate is a well known term in the art and refers to an aqueous solution containing plasmid, RNA, proteins (and usually residual amounts of chromosomal DNA) which is obtained after lysis of host cells and the separation of the cell debris, usually by filtration or centrifugation. Plasmid in the cleared lysate is usually a mixture of supercoiled and open circular plasmid.

The host cells containing plasmid are preferably bacteria, preferably Escherichia coli. Two methods are commonly used in the art for producing a cleared lysate from bacteria. Both methods comprise the steps of lysing the host cells, precipitating the chromosomal DNA, and removing the precipitated chromosomal DNA and cell debris. In the alkaline lysis method (Birnboim, 1979, Nucleic Acids Research, 7, 1513-23), host cells are lysed using an alkaline detergent solution. Chromosomal DNA is precipitated by neutralizing the lysed cell solution. The precipitated chromosomal DNA and cell debris is removed by filtration or centrifugation. In the boiling preparation method (Holmes, 1981, Analytical Biochemistry, 114, 193-7), host cells are lysed using lysozyme. Chromosomal DNA is precipitated by a brief heating step. The precipitated chromosomal DNA and cell debris is removed by centrifugation. The preferred method for preparing a cleared lysate is the alkaline lysis method.

After preparing the cleared lysate, the plasmid in the cleared lysate is optionally further purified prior to the enzymatic steps of the invention. Further purification can be accomplished by numerous known methods, such as organic solvent extraction, precipitation, ribonuclease incubation, chromatography, or combination. Further purification may be advantageous in several ways. First, further purification may result in plasmid in a buffer which is better suited for the enzymatic steps. Second, further purification may allow more efficient and reliable enzymatic reactions of the invention, by removing contaminants (such as protein and RNA) which might inhibit the enzymatic reactions. Further purification may unintentionally convert a small amount of supercoiled plasmid from the cleared lysate to open circular form. This unintentional conversion is the consequence of the inherent instability of supercoiled plasmid.

### The Enzymatic Steps of the Invention

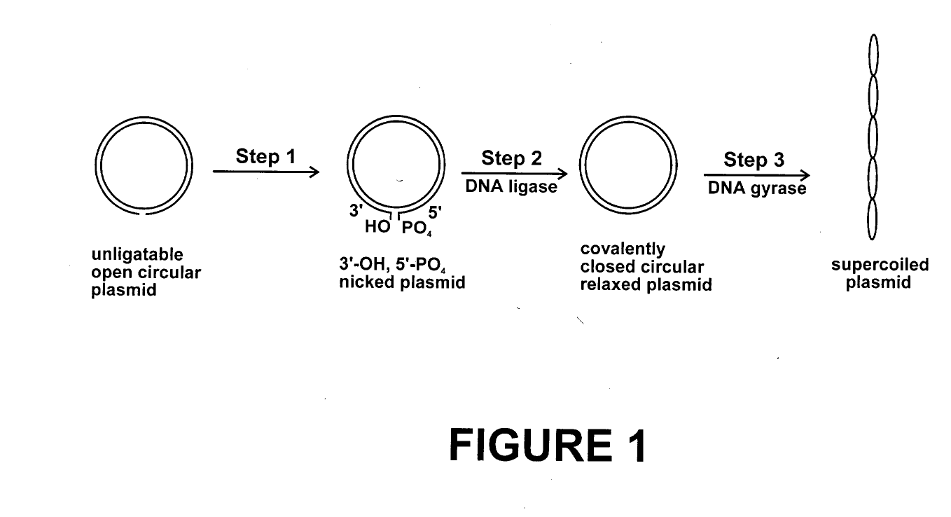

The method of the invention comprises three enzymatic steps, illustrated in FIG. 1.

Step 1: Conversion of Unligatable Open Circular Plasmid to 3′-hydroxyl, 5′-phosphate Nicked Plasmid.

In the first step of the invention, unligatable open circular plasmid is converted to 3′-hydroxyl, 5′-phosphate nicked plasmid. Unligatable open circular plasmid is defined as open circular plasmid which is not 3′-hydroxyl, 5′-phosphate nicked plasmid. This step can be accomplished in many ways, using enzymes in the art of DNA repair.

Mode 1: In one conversion method, denoted mode 1, the unligatable open circular plasmid is 3′-phosphate, 5′-hydroxyl nicked plasmid. This is converted to 3′-hydroxyl, 5′-phosphate nicked plasmid by incubation with the enzymes 3′-phosphatase and polynucleotide kinase. 3′-phosphatase converts the 3′-phosphate to 3′-hydroxyl. Polynucleotide kinase, in the presence of cofactor (usually ATP), converts the 5′-hydroxyl to 5′-phosphate. The result of the enzyme incubations is 3′-hydroxyl, 5′-phosphate nicked plasmid. The incubations with 3′-phosphatase and polynucleotide kinase are preferably performed simultaneously, but can also be performed sequentially in any order.

Mode 2: In a second preferred conversion method, denoted mode 2, the unligatable open circular plasmid may be nicked or gapped, and the termini may have almost any functional group. This unligatable open circular plasmid is converted to 3′-hydroxyl, 5′-phosphate nicked plasmid by incubation with the enzyme DNA polymerase in the presence of deoxynucleoside triphosphate substrates (dNTPs). Preferably, the polymerase is DNA polymerase I, with both 3′-5′ and 5′-3′ exonuclease activities. The 5′-3′ exonuclease activity of DNA polymerase I advantageously converts some 5′ termini that lack a 5′-phosphate to a 5′-phosphate terminus. This activity is also known as nick translation.

For some unligatable open circular plasmid, the 3′ terminus may be blocked by a functional group which impairs (completely or partially) the ability of DNA polymerase to extend the primer. This 3′ blocking group may be the result of DNA damage, such as free radical damage. In this case, a 3′ deblocking enzyme can remove the 3′ blocking group and produce a 3′-hydroxyl terminus. The resulting 3′-hydroxyl terminus can then be extended by DNA polymerase.

One useful preferred deblocking enzyme is exonuclease III. Exonuclease III converts 3′-blocked open circular plasmid to 3′-hydroxyl gapped plasmid. This is accomplished by the 3′-5′ exonuclease activity of exonuclease III. The known 3′-phosphatase and apurinic/apyrimidinic (AP) endonuclease activities of exonuclease III also serve as a 3′ deblocking function. DNA polymerase I converts the resulting 3′-hydroxyl gapped plasmid to 3′-hydroxyl, 5′-phosphate nicked plasmid in the presence of deoxynucleoside triphosphate substrates. The incubations with exonuclease III and DNA polymerase I are preferably performed simultaneously, but can also be performed sequentially in the order exonuclease III followed by DNA polymerase I. A 3′-deblocking enzyme which is closely related to exonuclease III is endonuclease IV. Other AP endonucleases may also serve as 3′-deblocking enzymes.

Another useful deblocking enzyme is 3′-phosphatase. 3′-Phosphatase is useful if the 3′ terminus blocking group is 3′-phosphate. The literature reports that the ability of DNA polymerase I (or Klenow) to extend a 3′-phosphate terminus is impaired, but not completely inhibited (Zhang, 2001, Biochemistry, 40, 153-9). DNA polymerase I is able to remove the 3′-phosphate or terminal nucleotide to produce a 3′-hydroxyl terminus, but this removal ability is very poor. In contrast, the deblocking enzyme 3′-phosphatase efficiently converts 3′-phosphate blocked open circular plasmid to 3′-hydroxyl open circular plasmid. DNA polymerase I converts the resulting 3′-hydroxyl open circular plasmid to 3′-hydroxyl, 5′-phosphate nicked plasmid in the presence of deoxynucleoside triphosphate substrates. The incubations with 3′-phosphatase and DNA polymerase I are preferably performed simultaneously, but can also be performed sequentially in the order: 3′-phosphatase followed by DNA polymerase I.

Other deblocking enzymes can be used for mode 2, provided that they convert the blocked 3′ terminus to a 3′ hydroxyl terminus. The deblocking enzyme may be selected from many known DNA repair enzymes, such as exonucleases, endonucleases (such as endonuclease IV), and phosphatases. More than one deblocking enzymes may be used for step 1. Repair enzymes may also be used to convert the 5′ terminus to a 5′-phosphate. Examples include polynucleotide kinase and 5′-3′ exonucleases.

Other Modes: The inventor has offered two general methods (modes 1 and 2) for performing step 1. It will be appreciated that any method for converting unligatable open circular plasmid to 3′-hydroxyl, 5′-phosphate nicked plasmid may be used in the invention. New methods for performing step 1 may be constructed from the many enzymes in the art of DNA repair.

Step 2: Conversion of 3′-hydroxyl, 5′-phosphate Nicked Plasmid to Relaxed Covalently Closed Circular Plasmid.

In the second step of the invention, the 3′-hydroxyl, 5′-phosphate nicked plasmid, derived from step 1, is converted to relaxed covalently closed circular (ccc) plasmid. This is accomplished by incubation with the enzyme DNA ligase in the presence of DNA ligase nucleotide cofactor.

Step 3: Conversion of Relaxed ccc Plasmid to Negatively Supercoiled Plasmid.

In the third step of the invention, the relaxed ccc plasmid, derived from step 2, is converted to negatively supercoiled plasmid. This is accomplished by incubation with the enzyme DNA gyrase in the presence of DNA gyrase nucleotide cofactor (usually ATP).

### Performing the Steps of the Invention

The three enzymatic steps of the invention are preferably performed simultaneously in a single combined incubation step, using an enzyme mixture. For mode 1, the enzyme mixture comprises 3′-phosphatase, polynucleotide kinase, DNA ligase, and DNA gyrase. For mode 2, the enzyme mixture comprises DNA polymerase I, DNA ligase, and DNA gyrase. The mode 2 mixture can further comprise one or more 3′ deblocking enzymes, such as exonuclease III or 3′-phosphatase. By using one combined incubation step, open circular plasmid unintentionally generated during the incubation step (for example by an enzyme contaminant) is converted to supercoiled plasmid. The three enzymatic steps of the invention can also be performed sequentially in the order: step 1, step 2, and step 3. Alternatively, steps 1 and 2 may be performed simultaneously, followed by step 3. Alternatively, step 1 may be performed, followed by steps 2 and 3 simultaneously.

Both modes in step 1 may be employed, sequentially or simultaneously. For example, modes 1 and 2 may be combined in a single combined incubation step comprising the enzyme mixture 3′-phosphatase, polynucleotide kinase, DNA polymerase I, DNA ligase, and DNA gyrase.

The enzymatic steps of the invention can be performed with intermediate purification of plasmid. For example, after step 2, plasmid could be purified by chromatography. The purified plasmid could subsequently be incubated with DNA gyrase for conversion to supercoiled form (step 3). Preferably, the enzymatic steps of the invention are performed without intermediate purification. That is, step 2 is preferably performed without prior purification of 3′-hydroxyl, 5′-phosphate plasmid after step 1. Step 3 is preferably performed without prior purification of ccc plasmid after step 2.

If the optimal incubation conditions, such as temperature or pH or buffer conditions, differ for the enzymes in the method, it may be advantageous to perform the enzymatic steps sequentially. For example, in mode 1, assume that polynucleotide kinase, 3′-phosphatase, and DNA ligase have an optimal incubation temperature of 37 degrees, and DNA gyrase is derived from a thermophile with an optimal incubation temperature of 75 degrees. In this case, step 1 and step 2 are performed at 37 degrees. The temperature is then increased to 75 degrees for the step 3 DNA gyrase incubation.

In some cases, it may be useful to perform the DNA gyrase incubation after completing the DNA ligase step, in order to reduce unproductive ATP hydrolysis by DNA gyrase. For example, a plasmid preparation may contain a high percentage of open circular plasmid. The net effect of DNA gyrase on open circular plasmid is unproductive hydrolysis of ATP. Completing the DNA ligase step prior to the DNA gyrase step avoids this problem. Preferably, however, the DNA ligase and DNA gyrase incubation is performed simultaneously.

### The Enzymes

Within the context of the invention, 3′-phosphatase and polynucleotide kinase enzymes should be active on open circular plasmid substrate. To the inventor's knowledge, 3′-phosphatase and polynucleotide kinase exist only in eukaryotes. Polynucleotide kinase and 3′-phosphatase enzyme activities are sometimes found on a single polypeptide in some organisms, denoted polynucleotide kinase-3′-phosphatase (PNKP). PNKP is known in the art as a DNA repair enzyme, repairing single stranded breaks in double stranded DNA. PNKP has been characterized in numerous organisms, including rats, human, bovine, plasmodium, S. pombe, and mouse (Karimi-Busheri et al, 1998, Nucleic Acids Research, 26, 4395-4400). 3′-Phosphatase with no associated polynucleotide kinase activity has been characterized in the yeast Saccharomyces cereviseae and the plant Arabidopsis thaliana (Vance et al, 2001, J. Biol. Chem., 276, 15073-81). Polynucleotide kinase with no associated 3′-phosphatase could potentially be obtained by mutation of PNKP. In the invention, the polynucleotide kinase and 3′-phosphatase enzymes can be present on the same protein (PNKP) or on separate proteins. Preferably, the two enzymes are present on the same protein. One useful source of PNKP for the invention is from human.

A non-specific phosphatase, such as alkaline phosphatase could be used as the equivalent of 3′-phosphatase, provided that the non-specific phosphatase activity is removed prior to subsequent steps. In the mode 1, non-specific phosphatase should be removed prior to the kinase step to prevent ATP hydrolysis and dephosphorylation of the 5′-phosphate terminus. In mode 2, non-specific phosphatase should be removed prior to DNA polymerase incubation to prevent hydrolysis of dNTPs and the 5′-phosphate terminus. Inactivation of alkaline phosphatase could be accomplished by heating. Preferably however, mode 1 employs 3-phosphatase, an enzyme which is specific for the 3′-phosphate of open circular plasmid.

DNA polymerase is employed in mode 2 of step 1. DNA polymerase lacking both 3′-5′ and 5′-3′ exonuclease activities could potentially convert a tiny amount of unligatable open circular plasmid to 3′-hydroxyl, 5′-phosphate nicked plasmid. For example, Sequenase DNA polymerase, which has no exonuclease activity, could be used in the invention to fill gaps in open circular plasmid. However, the preferred polymerase is DNA polymerase I, an enzyme having both 3′-5′ and 5′-3′ exonuclease activities. Preferably the polymerase is substantially not strand displacing on a nicked plasmid template, but instead hydrolyzes the strand by its 5′-3 exonuclease activity. The inventor has observed that DNA polymerase I, in the presence of deoxynucleotide triphosphate substrate, converts most of the open circular plasmid to 3′-hydroxyl, 5′-phosphate nicked plasmid. DNA polymerase I is likely ubiquitous in nature. One useful source of DNA polymerase I for the invention is from E. coli.

Exonuclease III is a known DNA repair enzyme, which is useful with DNA polymerase I in deblocking the 3′ terminus of 3′ blocked open circular plasmid. Exonuclease III, or closely related 3′-deblocking enzymes, is likely ubiquitous in nature. Exonuclease III has three activities, all of which may serve a deblocking function: 3′-5′ exonuclease activity, 3′-phosphatase activity, and apurinic/apyrimidinic (AP) endonuclease activity. Some organisms, such as Thermotoga maritima, do not appear to have exonuclease III in their genomes, and instead use the DNA repair enzyme endonuclease IV as a 3′ deblocking enzyme. One useful source of exonuclease III for the invention is from E. coli.

DNA ligase is ubiquitous in nature. DNA ligases from bacteriophage, viruses, eukaryotes, archaebacteria, and some eubacteria require adenosine triphosphate (ATP) as the cofactor. DNA ligases from eubacteria, such as E. coli, usually require nicotinamide adenine dinucleotide (NAD) as the cofactor. The invention can utilize DNA ligase from any source, provided that it is capable of ligating 3′-hydroxyl, 5′-phosphate nicks. It will be appreciated that equivalent cofactors could be used. For example, dATP could be used in place of ATP for some ligases. Preferably, the DNA ligase used in the invention requires ATP cofactor. One useful source of DNA ligase for the invention is from bacteriophage T4.

DNA gyrase is ubiquitous in eubacteria and has been isolated in some archeabacteria. This enzyme is involved in DNA replication. DNA gyrase converts relaxed ccc plasmid to negatively supercoiled plasmid in the presence of ATP or equivalent nucleotide. The invention may employ DNA gyrase from any source, provided that it converts relaxed ccc to supercoiled plasmid. One useful source of DNA gyrase for the invention is from E. coli. An especially useful source of DNA gyrase could be Vibrio cholera. Vibrio cholera DNA gyrase is reported to be unable to catalyze the reverse reaction (Mukhopadhyay et al, 1991, Biochemical J, 280, 797-800).

The DNA gyrase incubation step is preferably performed in the absence of topoisomerase I, which converts supercoiled plasmid to relaxed ccc plasmid. The presence of topoisomerase I during the DNA gyrase incubation could reduce the extent of supercoiling by DNA gyrase. It will be appreciated that enzyme purity is rarely absolute. Topoisomerase I may be considered absent, in a functional sense, if it is present at such a low level that it does not significantly affect the extent of supercoiling by DNA gyrase. The DNA gyrase incubation step could be performed in the presence of an inhibitor specific for topoisomerase I, reducing the detrimental effect of topoisomerase I.

The invention could also employ reverse DNA gyrase instead of DNA gyrase. Reverse DNA gyrase is found in many thermophilic bacteria. Reverse DNA gyrase converts relaxed ccc plasmid to positively supercoiled plasmid. The use of reverse DNA gyrase in the invention would produce a plasmid preparation of positively supercoiled plasmid. Preferably however, the invention employs DNA gyrase, as negatively supercoiled plasmid is known to be biologically active in human cells

### Optional Nucleotide Cofactor Regeneration

Several enzymes in the invention require nucleotide cofactors. DNA gyrase requires ATP for activity, generating ADP as the nucleotide cofactor by-product. Polynucleotide kinase requires ATP for activity, generating ADP as the nucleotide cofactor by-product. DNA ligase requires ATP (or NAD) for activity, generating AMP (or NMP) as the nucleotide cofactor by-product. For some enzyme incubations, very little ATP will be consumed. However, in some circumstances, a substantial amount of ATP could be consumed during the enzymatic reactions, and the ATP concentration may decline to undesirably low concentrations. This could possibly occur if there is a large amount of nicked plasmid, or if the initial ATP concentration is low. A large decline in ATP concentration may slow the enzymatic reactions. In such situations, it may be optionally desirable to maintain the ATP concentration at a constant optimal level. This is accomplished by enzymatically converting the nucleotide cofactor by-product back to nucleotide cofactor during the incubation step. The use of ATP regeneration during enzymatic incubations is well established in the art (Hinton et al, 1979, Nucleic Acids Res, 7, 453-64). The result of this method is maintaining a constant optimal concentration of nucleotide cofactor, avoiding any potential problem caused by a decline in ATP concentration.

In step 3 of the invention, the DNA gyrase incubation step generates ADP as the nucleotide cofactor by-product. Optionally, ADP can be converted back to ATP during the DNA gyrase incubation using a kinase enzyme and a high energy phosphate donor. The preferred kinase and phosphate donor is pyruvate kinase and phosphoenolpyruvate (PEP). The pyruvate kinase and PEP is coincubated with DNA gyrase to maintain a constant ATP concentration. Another kinase and high energy phosphate donor example is creatine kinase and creatine phosphate. This method can also be employed in the polynucleotide kinase incubation step of mode 1, converting ADP, the nucleotide cofactor by-product, back to ATP.

In step 2 of the invention, the DNA ligase incubation step generates AMP as the nucleotide cofactor by-product. Optionally, AMP can be converted back to ATP during the DNA ligase incubation using a mixture of adenylate kinase, pyruvate kinase, and PEP. Adenylate kinase converts AMP to ADP in the presence of ATP. Pyruvate kinase and PEP convert ADP to ATP. Adenylate kinase, pyruvate kinase, and PEP are coincubated with DNA ligase to maintain a constant ATP concentration. If the cofactor for DNA ligase is NAD, the nucleotide cofactor by-product nicotinamide monophosphate (NMP) can be converted back to NAD by the enzyme nicotinamide adenylyltransferase. AMP generated by the latter enzyme could be converted back to ATP as described.

Pyrophosphate is generated as a by-product of the DNA ligase and the DNA polymerase reaction. A build up in the pyrophosphate concentration may slow these reactions. Optionally, it may be desirable to include the enzyme inorganic pyrophosphatase during the DNA ligase or the DNA polymerase incubation. Hydrolysis of pyrophosphate to phosphate by inorganic pyrophosphatase avoids this potential problem.

In mode 2, the DNA polymerase I incubation step generates dNMP by-products. The dNMP by-products could optionally be enzymatically converted back to dNTPs during the DNA polymerase I incubation. This is accomplished using the enzymes cytidylate kinase, thymidylate kinase, adenylate kinase, guanidylate kinase, and nucleoside diphosphate kinase. For example, dCMP is converted to dCDP by cytidylate kinase, which is converted to dCTP by nucleoside diphosphate kinase.

In one embodiment of mode 1, the enzymatic steps are performed in one incubation step using a mixture of 3′-phosphatase, polynucleotide kinase, DNA ligase, and DNA gyrase. If the latter three enzymes require ATP cofactor, then adding adenylate kinase, pyruvate kinase, and PEP to this incubation step would maintain a constant ATP concentration.

Nucleotide cofactor regeneration may be especially advantageous at high concentrations of DNA gyrase and DNA ligase. DNA gyrase is known to hydrolyze ATP, even in the absence of DNA substrate. In addition, the inventor believes that DNA ligase also slowly hydrolyzes ATP to AMP in the absence of DNA substrate. At high enzyme concentrations, ATP hydrolysis could be rapid. The use of an enzymatic system to convert nucleotide cofactor by-product back to the nucleotide cofactor avoids a decline in ATP concentration.

The use of enzymes for regenerating nucleotide cofactor from their nucleotide by-product is optional in the invention. To the inventor's knowledge, the use of nucleotide cofactor regeneration for the enzymes DNA ligase and polynucleotide kinase is not known in the literature.

### Optional Additional Exonuclease Step

An optional additional exonuclease incubation step may be performed to selectively hydrolyze residual linear chromosomal DNA contamination without hydrolyzing plasmid. The selective conversion of linear chromosomal DNA to nucleotides or small oligonucleotides facilitates their subsequent removal from plasmid. The use of exonucleases for plasmid purification is well established (Isfort, 1992, Biotechniques, 12, 800-3). It will be appreciated that the selectivity of the exonuclease need not be absolute. A small loss of plasmid due to lack of absolute specificity by an exonuclease may be acceptable for the user. The result of this step is a reduction in the chromosomal DNA contamination in the final plasmid preparation. One or more exonucleases may be used for this incubation step.

The composition of the exonucleases depends on when the exonuclease step is performed. If the exonuclease step is performed prior to the conversion of open circular plasmid to relaxed ccc plasmid, the exonucleases should selectively degrade the linear chromosomal DNA, substantially without degrading open circular plasmid, relaxed ccc plasmid, or supercoiled plasmid. Several such exonucleases are known in the art, including exonuclease I, lambda exonuclease, and ATP dependent exonuclease. In addition, deblocking enzymes which are also exonucleases may serve a dual function of hydrolyzing chromosomal DNA. If the exonuclease step is performed after open circular plasmid is converted to relaxed ccc plasmid, the exonucleases should selectively degrade linear chromosomal DNA, substantially without degrading either relaxed ccc plasmid or supercoiled plasmid. Examples of such exonucleases include those listed above and also include exonuclease III.

The preferred exonuclease is ATP dependent exonuclease, also known as recBCD. ATP dependent exonuclease hydrolyzes linear chromosomal DNA to small oligonucleotides. This enzyme requires the cofactor ATP, generating ADP as the nucleotide cofactor by-product. The use of ATP dependent exonuclease is synergistic in the invention. The ATP dependent exonuclease incubation step could be performed in the presence of a kinase enzyme and high energy phosphate donor which converts ADP nucleotide cofactor by-product back to ATP, as described previously. In one synergistic embodiment, the enzymatic steps are performed in a single incubation step using a mixture of the enzymes: DNA polymerase I, DNA ligase, DNA gyrase, ATP dependent exonuclease, and optionally regenerating enzymes which convert AMP and ADP (the nucleotide cofactor by-products) back to ATP (such as adenylate kinase, pyruvate kinase, and PEP). To the inventor's knowledge, the use of ATP regeneration during ATP dependent exonuclease digestion is not known in the prior art.

It is conceivable that the oligonucleotide products of ATP dependent exonuclease digestion could be polymerized by DNA ligase. However, the inventor has not observed polymerization experimentally. The inventor postulates that the oligonucleotide products are poor substrates for DNA ligase. If polymerization does occur to a significant extent, the problem could be solved by: (a) increasing the concentration of ATP dependent exonuclease, (b) using a DNA ligase which is unable to ligate blunt ends, such as E. coli DNA ligase, (c) adding an additional exonuclease, such as exonuclease I, to hydrolyze the oligonucleotides to nucleotides, or (d) performing the exonuclease digestion step after the DNA ligase incubation step.

The invention optionally could further comprise a ribonuclease digestion step to hydrolyze residual RNA. Ribonuclease incubation step could be performed at any step in the invention. The ribonuclease incubation step could be performed as an isolated step or simultaneously with an enzymatic step in the invention. The use of ribonuclease is well established in the art of plasmid purification. Preferably, the ribonuclease is ribonuclease I.

Undesired plasmid may be removed by selective restriction endonuclease digestion. If two or more plasmids are present in a cleared lysate, usually only one plasmid is the desired product. For example, a host cell may contain two different plasmids. Alternatively, two different plasmids could be generated from one plasmid in a clarified lysate by incubation with a recombinase. The resulting selectively linearized undesired plasmid could be further digested by the exonuclease incubation. It will be appreciated that the use of restriction enzyme in this manner does not involve linearization of the plasmid of interest.

### Optional Additional Potent Decatenase Step

One potential problem, not observed by the inventor, is formation of catenanes. A catenane is formed by interlocking of two plasmid molecules. DNA gyrase could potentially catalyze catenane formation, where both plasmids are still supercoiled. The level of catenane formation should be very small. It is known in the art that DNA gyrase is a very weak catenase. Also, DNA gyrase is known to have weak decatenase activity. Thus, DNA gyrase could decatenate any catenanes, thereby limiting accumulation. The inventor has not observed any significant catenation. If catenation does occur, the amount of catenane formed is probably insignificant for most applications.

If catenane formation does occur to an undesirable extent, as determined by the user, then catenane formation can be reduced by several methods. In one method, the DNA gyrase incubation step could be performed at a lower plasmid concentration or performed in a manner that minimizes plasmid aggregation. In a second method, a DNA gyrase with stronger decatenase activity can be employed, such as mycobacterial smegmatis DNA gyrase. In a third method, catenation can be reduced or eliminated by an optional additional incubation step with a potent decatenase enzyme. The potent decatenase incubation step is preferably performed simultaneously with the DNA gyrase incubation, but could be performed after the DNA gyrase incubation step. Topoisomerase III and topoisomerase IV are known in the art as potent decatenases. Both decatenases relax supercoiled plasmid at a slow rate. Therefore, these potent decatenases should be used at a minimal concentration, to effect decatenation and to minimize supercoiled relaxation. The preferred potent decatenase is topoisomerase IV.

Both known potent decatenases convert ATP nucleotide cofactor to ADP (the nucleotide cofactor by-product). The optional potent decatenase step could be performed in the presence of an enzyme and high energy phosphate donor to convert ADP back to ATP. An example, described earlier, is pyruvate kinase and PEP.

### Plasmid Recovery

After the enzymatic steps of the invention, the resulting plasmid can be used directly in some applications without further purification. For other applications, additional purification may be optionally desirable to remove the buffer salts, enzymes, nucleotides, and possibly exonuclease digestion products. This can be accomplished by many known methods, such as organic solvent extraction, chromatography (gel filtration, anion exchange, hydrophobic interaction, reverse phase), precipitation, ultrafiltration, ultracentrifugation, electrophoresis, or combination.

In one advantageous embodiment of the invention, plasmid from a cleared lysate is purified chromatographically prior to the enzymatic steps of the invention. After the enzymatic steps of the invention, the plasmid product is purified using the same chromatographic column, as a final polishing step. The chromatographic column in this case is preferably an anion exchange column, such as a commercially available anion exchange columns for plasmid purification (Qiagen, Macherey-Nagel).

Applications for the plasmid include transformation into recipient competent cells, in vitro and in vivo. The invention is especially suited for producing plasmid for human therapeutic use. When used in combination with the optional exonuclease step, the final plasmid product has a high percentage supercoiled plasmid and a low percentage of chromosomal DNA contamination.

### Repair Enzymes and Accessory Proteins

The repair of single stranded breaks in double stranded DNA is an essential function of the DNA repair system of all living organisms. Numerous repair enzymes and accessory proteins are described in the art of DNA repair which facilitate the repair of single stranded breaks of all types. Such enzymes and accessory proteins could be used in step 1 of the invention to accelerate or improve the conversion of unligatable open circular plasmid to ccc plasmid. For example, AP endonucleases could be used to remove 3′-terminal blocking lesions. 5′-3′ exonucleases could be used to remove 5′ blocking groups. Protein XRCC1 and poly(ADP-ribose) polymerase 1 could be employed to accelerate the repair of single stranded breaks catalyzed by DNA ligase and PNKP. Protein HU in prokaryotes has been implicated in assisting repair of single stranded breaks.

### Enzyme Reuse

In one embodiment of the invention, one or more of the enzymes could be covalently attached to a solid support. The resulting enzyme-solid support could be packed in a chromatography column, producing a enzyme column. An enzyme column could be made for each enzyme in the method separately. Alternatively, one enzyme column could contain a mixture of enzymes to completely convert unligatable open circular plasmid to supercoiled plasmid. Plasmid solution is pumped through the column or series of columns, converting unligatable open circular plasmid to supercoiled plasmid. Column eluate could be recycled through the column(s) as needed until all unligatable open circular plasmid is converted to supercoiled plasmid. A single enzyme columns could be reused multiple times to prepare multiple plasmids. Preferably, however, the enzymes in the invention are not attached to a solid support and are free in solution.

For bulk scale plasmid preparations, a large quantity of the enzymes in the invention may be needed. Producing a large quantity of enzymes may be expensive. In this case, it may be advantageous to recover the enzymes after the incubation, so that the enzymes could be reused for subsequent plasmid preparations. To recover the enzymes for reuse, the enzyme must be separated from the plasmid. This could be performed by using affinity chromatography if the enzymes have an affinity tag, such as polyhistidine. This could also be performed using classical chromatography, such as anion or cation exchange, which would separate the plasmid from the enzymes. If the enzymes are recovered after the incubation, the full enzyme activity should be maintained during the incubation. This could be accomplished by lowering the incubation temperature slightly or by adding known enzyme stabilizers, such as glycerol, Triton X-100, spermidine, bovine serum albumin, or dithiothreitol.

In one advantageous embodiment, the enzymes of the invention are thermostable and are derived from a thermophilic organism. Recombinant thermostable enzymes are readily purified from E. coli, since E. coli proteins are unstable at higher temperatures. For example, some or all of the enzymes could be derived from the thermophile Bacillus stearothermophilus or Thermotoga maritima. The incubations in the invention could be performed at temperatures between 50 degrees and 75 degrees. Alternatively, some or all of the enzymes could be derived from a thermophilic eukaryote, such as thermomyces lanuginosus. Thermostable enzymes would maintain their full activity during the incubation, optionally allowing reuse for subsequent incubations if desired.

### Steps Preferably not Performed

After preparing a cleared lysate, the cleared lysate usually comprises supercoiled plasmid, in addition to open circular plasmid. After preparing the cleared lysate, the supercoiled plasmid is preferably not purposefully modified prior to the enzymatic steps of the invention. Purposeful modification is usually a quantitative conversion, in which most of the material is converted. Preferably, after preparing a cleared lysate and prior to the enzymatic steps in the invention, supercoiled plasmid from the cleared lysate is not purposefully converted to open circular plasmid, for example by intentional free radical nicking, incubation with a nickase such as NBstBI, or DNase I nicking. Preferably, after preparing a cleared lysate and prior to the enzymatic steps in the invention, supercoiled plasmid from the cleared lysate is not purposefully converted to ccc relaxed plasmid, for example by incubation with topoisomerase I or incubation with DNA ligase+AMP. Preferably, after preparing a cleared lysate and prior to the enzymatic steps in the invention, supercoiled plasmid (or open circular plasmid) is not purposefully converted to linear form, for example by restriction digestion.

Preferably, after preparing the cleared lysate and prior to the enzymatic steps in the invention, the open circular plasmid is not purposefully converted to single stranded circular DNA, for example by heating.

Preferably, after preparing a cleared lysate and prior to the enzymatic steps in the invention, the nucleotide sequence of the plasmid is not modified.

Preferably, after preparing a cleared lysate, the enzymatic steps of the invention are performed without in vitro plasmid replication and without prior in vitro plasmid replication. “In vitro plasmid replication” is defined in the invention as enzymatic production of daughter plasmid molecules (either partial or full production) from a parent plasmid in vitro. Partial production of daughter molecules on some plasmids produces a theta structure on electron microscopic observation. Partial production of daughter molecules by rolling circle replication results in production of single stranded molecules from the parent plasmid. An example of in vitro plasmid replication is described by Funnel et al (J. Biol. Chem, 1986, 261, 5616-24).

Preferably, the enzymatic steps of the invention are performed without an incubation step with a primase enzyme, which forms primers for synthesis of daughter strands of plasmid.

Preferably, the enzymatic steps of the invention are performed without increasing the amount of plasmid material in vitro during the steps of the invention, where conversion of gapped plasmid in a cleared lysate to nicked plasmid is not considered increasing the amount of plasmid.

Preferably, the enzymatic steps of the invention are performed substantially without using a strand displacing DNA polymerase, which generates displaced single stranded DNA.

Preferably, the enzymatic steps of the invention are performed in a manner to minimize or avoid in vitro recombination events. For example, the enzymatic steps are preferably performed in the absence of recA protein or in the absence of single stranded DNA binding protein, both of which promote recombination events.

Preferably, the unligatable open circular plasmid employed in the enzymatic steps of the invention is derived from (i) unligatable open circular plasmid which exists in host cells immediately prior to lysis, (ii) supercoiled plasmid in host cells which is unintentionally converted to unligatable open circular plasmid in the preparation of the cleared lysate, or (iii) supercoiled plasmid in the cleared lysate which is unintentionally converted to unligatable open circular plasmid after further plasmid purification steps prior to the enzymatic steps in the invention. As discussed, unintentional conversion is the consequence of the inherent instability of plasmid to DNA damage. Preferably, the unligatable open circular plasmid is not derived from an in vitro enzymatic reaction which produces unligatable open circular plasmid from non-plasmid DNA. For example, unligatable open circular plasmid is preferably not derived from single stranded circular DNA (non-plasmid), which is converted to unligatable open circular plasmid by an in vitro enzymatic reaction.

It will be appreciated that the enzymatic steps of the invention are not perfect. Unintentional plasmid modification may occur. This unintentional conversion may be the result of enzyme impurities. For example, nuclease contamination may convert supercoiled plasmid to open circular plasmid. Unintentional conversion may also result from the side reactions of the inherent activity of the enzymes employed in the invention. Several examples illustrate this point. (1) DNA polymerase I may convert a small amount of open circular plasmid to single stranded circular DNA as the result of 3′-5′ exonuclease activity. This conversion is not considered purposeful, since the purpose of DNA polymerase is producing 3′-hydroxyl, 5′-phosphate nicked plasmid. (2) DNA ligase or DNA gyrase may convert a small amount of supercoiled plasmid to relaxed covalently closed circular plasmid. This conversion is not considered purposeful, since the purpose of these enzymes is to convert 3′-hydroxyl, 5′-phosphate nicked plasmid to supercoiled plasmid. (3) ATP dependent exonuclease may convert a tiny amount of gapped plasmid to linear form, by hydrolysis of the single stranded region of the gapped plasmid. This conversion is not considered purposeful, since the purpose of ATP dependent exonuclease is hydrolysis of chromosomal DNA. (4) DNA polymerase I may produce a tiny amount of displaced strand as a side reaction, despite the fact that it possesses 5′-3 exonuclease activity. This is not considered purposeful strand displacement, since the purpose of DNA polymerase I is nick translation. (5) An AP endonuclease (such as exonuclease III) may convert a small amount of supercoiled plasmid to nicked plasmid, if the supercoiled plasmid contains an abasic, site. This is not be considered purposeful nicking, since the purpose of the AP endonuclease is the repair of open circular plasmid.

### Enzyme Reagents

Performing the method of the invention is facilitated by using premixed enzyme reagents. One useful enzyme reagent comprises polynucleotide kinase, 3′-phosphatase, DNA ligase, and DNA gyrase. Preferably, polynucleotide kinase and 3′-phosphatase are present on the same polypeptide (the enzyme PNKP). The preferred enzyme reagent comprises DNA polymerase I, DNA ligase, and DNA gyrase. Preferably, this reagent does not comprise additional enzymes (i) which result in vitro plasmid replication and (ii) which result in conversion of single stranded circular DNA to open circular DNA without using a synthetic primer. Examples of such additional enzymes may include primase, single stranded DNA binding protein, or DNA polymerase III. The preferred reagent can further comprise one or more 3′ terminus deblocking enzymes. The 3′-deblocking enzyme may be 3′-phosphatase, exonuclease III, other deblocking enzyme(s), or combination.

To the inventor's knowledge, enzyme reagents comprising 3′-phosphatase do not exist in nature. According to the literature, DNA gyrase exists only in prokaryotes; whereas 3′-phosphatase exists only in eukaryotes.

Preferably, the enzyme reagent does not comprise topoisomerase I. The enzyme reagent can further comprise one or more of the following enzymes: (1) regenerating enzymes to convert the nucleotide by-product of cofactor back to cofactor, (2) inorganic pyrophosphatase, (3) one or more exonucleases to selectively hydrolyze residual chromosomal DNA, such as ATP dependent exonuclease, (4) topoisomerase IV, and (5) ribonuclease to hydrolyze residual RNA contamination, such as RNase One.

The enzymes in the reagents could be produced using recombinant DNA technology as genetic fusions with affinity fusion protein tags to facilitate purification. For example, the enzymes could be fused to glutathione-S-transferase or polyhistidine and purified by affinity chromatography on glutathione agarose or nickel chelating resin respectively. The enzymes could be purified to lower endotoxin contamination to low levels. Thus, the enzyme incubation would not contaminate the plasmid with endotoxin. The enzyme reagents could be supplied in lyophilized form or in a solution, such as a buffered 50% glycerol solution.

### Advantages Over Prior Art

The method of the invention differs in a fundamental manner from all prior art methods for purifying supercoiled plasmid. All prior art methods are based on excluding open circular plasmid from the final plasmid preparation. The invention is based on including open circular plasmid in the final plasmid preparation. This is accomplished by enzymatically converting open circular plasmid to supercoiled plasmid.

As a consequence of the inclusion principle, one advantage of the invention over prior art methods is increased supercoiled plasmid yield. For example, assume that a plasmid preparation has 25% open circular plasmid and 75% supercoiled plasmid. Using prior art methods, the theoretical maximum yield of supercoiled plasmid is 75% of the starting plasmid. In the invention, the theoretical maximum yield of supercoiled plasmid is 100% of starting plasmid. The invention removes concern about nicking damage in the initial plasmid processing, as any nicked plasmid will be converted to supercoiled plasmid. The invention is especially useful for preparing large plasmids, which tend to have a higher percentage of open circular plasmid due to their larger size.

To the inventor's knowledge, DNA gyrase, DNA ligase, DNA polymerase I, polynucleotide kinase, and 3′-phosphatase have never been applied in the field of plasmid purification. The use of these enzymes breaks new ground in the art of plasmid preparation.

In addition, the invention offers a solution to a previously unrecognized problem in the art of plasmid preparation—the extent of supercoiling. The extent of supercoiling of plasmid can vary from batch to batch and from different fermentation conditions. The extent of supercoiling may have an effect on the biological activity of the plasmid. For example, a plasmid preparation which has a low extent of supercoiling may be less bioactive than desired. In the literature, it is reported that extent of supercoiling of plasmid in bacteria is not at its thermodynamic maximum (Cullis et al, 1992, Biochemistry, 31, 9642-6). This is due to the effect of topoisomerase I in the bacteria which relaxes supercoiled plasmid. Thus, the extent of supercoiling in bacteria is an equilibrium effect between DNA gyrase and topoisomerase I.

The invention solves this problem by incubation with DNA gyrase, preferably in the absence of topoisomerase I. The gyrase incubation in the invention could increase the extent of supercoiling. Plasmid could be supercoiled to its thermodynamic limit. The increased supercoiled state could create a more condensed plasmid molecule with potentially greater transformability. In summary, the DNA gyrase incubation step of the invention could convert all plasmid (including pre-existing supercoiled plasmid from the host) to a more highly supercoiled state.

The method of the invention is further illustrated by the following non-limiting examples.

### EXAMPLE 1

### Materials for the Examples

T4 DNA ligase and human PNKP were produced as fusion proteins with glutathione-S-transferase (GST) affinity tag as follows. The genes coding for these enzymes were amplified by the polymerase chain reaction. The genes were cloned into pGEX, a commercially sold expression vector (Amersham) so that the GST affinity tag was fused to the amino terminus of the enzyme. The fusion proteins were purified on glutathione-agarose according to the manufacturer's instructions. These fusion proteins are denoted GST-T4 DNA ligase and GST-PNKP.

A five kilobase plasmid in an E. coli host was purified using the alkaline lysis method as previously described (Maniatis et al, 1982, Molecular Cloning: A laboratory manual, Cold Spring Harbor Laboratory, 368-9). Agarose gel electrophoresis showed approximately 5% nicked plasmid and 95% supercoiled plasmid. This plasmid preparation, denoted p5 kb, was used in the subsequent examples.

A four kilobase plasmid in an E. coli host was purified using the alkaline lysis method. The plasmid preparation was further purified using established methods in the art to remove RNA contamination. Agarose gel electrophoresis showed approximately 20% nicked plasmid, 80% supercoiled plasmid, and some residual chromosomal DNA also likely present. This plasmid preparation, denoted p4 kb, was used in the subsequent examples. A six kilobase plasmid was prepared in a similar manner as p4 kb. Agarose gel electrophoresis showed approximately 10% nicked plasmid, 90% supercoiled plasmid, and some residual chromosomal DNA also likely present. This plasmid preparation, denoted p6 kb, was used in the subsequent examples.

To further illustrate the method, a fully nicked plasmid was prepared as follows. The p4 kb plasmid preparation, described above, was incubated with the nickase enzyme NBstBI (New England Biolabs) at 50 units/ml final concentration at 52 degrees for 1 hour. The reaction was extracted with phenol:CHCl₃, alcohol precipitated, and dissolved in alkaline phosphatase buffer. The plasmid was dephosphorylated by incubation with alkaline phosphatase. The reaction was extracted with phenol:CHCl₃, alcohol precipitated, and dissolved in TE buffer (10 mM Tris-Cl, 1 mM EDTA, pH 8.0). Agarose gel electrophoresis showed virtually 100% of the plasmid in the nicked form. This nicked plasmid contains mostly 3′-hydroxyl, 5′-hydroxyl nicks. Since the 5′ terminus of the nicks is dephosphorylated, DNA ligase alone cannot convert to this nicked plasmid to relaxed ccc plasmid. This preparation, denoted p4 kb-NBstBI-AP, was used in the subsequent examples to illustrate how the invention can convert a completely nicked plasmid preparation to a supercoiled plasmid preparation. In the prior art, a plasmid preparation containing 100% nicked plasmid would be discarded. The examples demonstrate that such a terribly nicked plasmid preparation instead could be converted to a useful supercoiled plasmid preparation.

### EXAMPLE 2

### Mode 1

A 10 μl reaction volume contained 1 μg p5 kb plasmid, 35 mM Tris-HCl, pH 7.5, 25 mM KCl, 4 mM MgCl₂, 2 mM dithiothreitol, 1.8 mM spermidine, 1 mM ATP, 6.4% glycerol, 0.1 mg/ml bovine serum albumin, 2.5 units DNA gyrase (E. coli), 2.8 μg GST-T4 DNA ligase, 1.4 μg GST-PNKP. This reaction was incubated at 37 degrees for 2 hours. After the incubation, the plasmid was analyzed by agarose gel electrophoresis. The gel showed high purity supercoiled plasmid, confirming conversion of most of the open circular plasmid to supercoiled plasmid.

The same incubation was performed using 5 μg of p4 kb plasmid. After the incubation, the plasmid was analyzed by agarose gel electrophoresis. The gel showed conversion of some of the open circular plasmid to supercoiled plasmid.

The same incubation was performed using 5 μg of p6 kb plasmid. After the incubation, the plasmid was analyzed by agarose gel electrophoresis. The gel showed conversion of most of the open circular plasmid to supercoiled plasmid.

The same incubation was performed using 5 μg of p4 kb-NBstBI-AP plasmid. After the incubation, the plasmid was analyzed by agarose gel electrophoresis. The gel showed conversion of most of the open circular plasmid to supercoiled plasmid.

### EXAMPLE 3

### Mode 1+ATP Dependent Exonuclease

A 10 μl reaction volume contained 1 μg p4 kb plasmid, 35 mM Tris-HCl, pH 7.5, 25 mM KCl, 4 mM MgCl₂, 2 mM dithiothreitol, 1.8 mM spermidine, 1 mM ATP, 6.4% glycerol, 0.1 mg/ml bovine serum albumin, 2.5 units DNA gyrase (E. coli), 2.8 μg GST-T4 DNA ligase, 1.4 μg GST-PNKP, 0.05 units PlasmidSafe (ATP dependent exonuclease, Epicentre). This reaction was incubated at 37 degrees for 2 hours. After the incubation, the plasmid was analyzed by agarose gel electrophoresis. The gel showed conversion of some of the open circular plasmid to supercoiled plasmid.

### EXAMPLE 4

### Mode 1+Topoisomerase IV

A 10 μl reaction volume contained 1 μg p4 kb plasmid, 35 mM Tris-HCl, pH 7.5, 25 mM KCl, 4 mM MgCl₂, 2 mM dithiothreitol, 1.8 mM spermidine, 1 mM ATP, 6.4% glycerol, 0.1 mg/ml bovine serum albumin, 2.5 units DNA gyrase (E. coli), 2.8 μg GST-T4 DNA ligase, 1.4 μg GST-PNKP, 0.08 picomoles topoisomerase IV (Bacillus subtilis). This reaction was incubated at 37 degrees for 2 hours. After the incubation, the plasmid was analyzed by agarose gel electrophoresis. The gel showed conversion of some of the open circular plasmid to supercoiled plasmid.

### EXAMPLE 5

### Mode 2

A 10 μl reaction volume contained 5 μg p4 kb plasmid, 35 mM Tris-HCl, pH 7.5, 25 mM KCl, 4 mM MgCl₂, 2 mM dithiothreitol, 1.8 mM spermidine, 1 mM ATP, 6.4% glycerol, 0.1 mg/ml bovine serum albumin, 2.5 units DNA gyrase (E. coli), 2.8 μg GST-T4 DNA ligase, 5 units DNA polymerase I (E. coli), 200 μM dATP, 200 μM dGTP, 200 μM dCTP, 200 μM dTTP. This reaction was incubated at 37 degrees for 2 hours. After the incubation, the plasmid was analyzed by agarose gel electrophoresis. The gel showed high purity supercoiled plasmid, confirming conversion of most of the open circular plasmid to supercoiled plasmid.

The same incubation was performed using 5 μg of p6 kb plasmid. After the incubation, the plasmid was analyzed by agarose gel electrophoresis. The gel showed conversion of most of the open circular plasmid to supercoiled plasmid.

The same incubation was performed using 5 μg of p4 kb-NBstBI-AP plasmid. After the incubation, the plasmid was analyzed by agarose gel electrophoresis. The gel showed conversion of most of the open circular plasmid to supercoiled plasmid.

### EXAMPLE 6

### Mode 2+ATP Regeneration+Pyrophosphatase

A 10 μl reaction volume contained 5 μg p4 kb plasmid, 35 mM Tris-HCl, pH 7.5, 25 mM KCl, 4 mM MgCl₂, 2 mM dithiothreitol, 1.8 mM spermidine, 1 mM ATP, 6.4% glycerol, 0.1 mg/ml bovine serum albumin, 2.5 units DNA gyrase (E. coli), 2.8 μg GST-T4 DNA ligase, 5 units DNA polymerase I (E. coli), 200 μM dATP, 200 μM dGTP, 200 μM dCTP, 200 μM dTTP, 0.05 units adenylate kinase (Sigma M5520), 0.05 units creatine kinase (Sigma C3755), 0.005 units inorganic pyrophosphatase (Sigma I1643), 5 mM creatine phosphate. This reaction was incubated at 37 degrees for 2 hours. After the incubation, the plasmid was analyzed by agarose gel electrophoresis. The gel showed high purity supercoiled plasmid, confirming conversion of most of the open circular plasmid to stipercoiled plasmid.

### EXAMPLE 7

### Mode 2+ATP Dependent Exonuclease

A 10 μl reaction volume contained 1 μg p4 kb plasmid, 35 mM Tris-HCl, pH 7.5, 25 mM KCl, 4 mM MgCl₂, 2 mM dithiothreitol, 1.8 mM spermidine, 1 mM ATP, 6.4% glycerol, 0.1 mg/ml bovine serum albumin, 5 units DNA gyrase (E. coli), 2.8 μg GST-T4 DNA ligase, 1.4 μg GST-PNKP, 0.05 units PlasmidSafe (ATP dependent exonuclease, Epicentre Technologies). This reaction was incubated at 37 degrees for 2 hours. After the incubation, the plasmid was analyzed by agarose gel electrophoresis. The gel showed high purity supercoiled plasmid, confirming conversion of virtually all open circular plasmid to supercoiled plasmid.

### EXAMPLE 8

### Mode 2+Exonuclease III Deblocking Enzyme

A 10 μl reaction volume contained 1 μg p4 kb plasmid, 35 mM Tris-HCl, pH 7.5, 25 mM KCl, 4 mM MgCl₂, 2 mM dithiothreitol, 1.8 mM spermidine, 1 mM ATP, 6.4% glycerol, 0.1 mg/ml bovine serum albumin, 2.5 units DNA gyrase (E. coli, Sigma), 2.8 μg GST-T4 DNA ligase, 5 units DNA polymerase I (New England Biolabs), 50 units exonuclease III (New England Biolabs). This reaction was incubated at 37 degrees for 2 hours. After the incubation, the plasmid was analyzed by agarose gel electrophoresis. The gel showed high purity supercoiled plasmid, confirming conversion of virtually all open circular plasmid to supercoiled plasmid.

The same incubation was performed using 5 μg of p4 kb-NBstBI-AP plasmid. After the incubation, the plasmid was analyzed by agarose gel electrophoresis. The gel showed high purity supercoiled plasmid, confirming conversion of virtually all open circular plasmid to supercoiled plasmid.

### EXAMPLE 9

### Mode 2+exonuclease III+ATP regeneration+pyrophosphatase

A 10 μl reaction volume contained 1 μg p4 kb plasmid, 35 mM Tris-HCl, pH 7.5, 25 mM KCl, 4 mM MgCl₂, 2 mM dithiothreitol, 1.8 mM spermidine, 1 mM ATP, 6.4% glycerol, 0.1 mg/ml bovine serum albumin, 2.5 units DNA gyrase (E. coli), 2.8 μg GST-T4 DNA ligase, 5 units DNA polymerase I (New England Biolabs), 50 units exonuclease III (New England Biolabs), 0.05 units adenylate kinase (Sigma M5520), 0.05 units creatine kinase (Sigma C3755), 0.005 units inorganic pyrophosphatase (Sigma I1643), 5 mM creatine phosphate. This reaction was incubated at 37 degrees for 2 hours. After the incubation, the plasmid was analyzed by agarose gel electrophoresis. The gel showed high purity supercoiled plasmid, confirming conversion of virtually all open circular plasmid to supercoiled plasmid.

The same incubation was performed using 5 μg of p4 kb-NBstBI-AP plasmid. After the incubation, the plasmid was analyzed by agarose gel electrophoresis. The gel showed high purity supercoiled plasmid, confirming conversion of virtually all open circular plasmid to supercoiled plasmid.

### EXAMPLE 10

### Mode 2+3′-phosphatase Deblocking Enzyme

A 10 μl reaction volume contained 5 μg p6 kb plasmid, 35 mM Tris-HCl, pH 7.5, 25 mM KCl, 4 mM MgCl₂, 2 mM dithiothreitol, 1.8 mM spermidine, 1 mM ATP, 6.4% glycerol, 0.1 mg/ml bovine serum albumin, 2.5 units DNA gyrase (E. coli), 2.8 μg GST-T4 DNA ligase, 1.4 μg GST-PNKP, 5 units DNA polymerase I (E. coli), 200 μM dATP, 200μM dGTP, 200 μM dCTP, 200 μM dTTP. This reaction was incubated at 37 degrees for 2 hours. After the incubation, the plasmid was analyzed by agarose gel clectrophoresis. The gel showed high purity supercoiled plasmid, confirming conversion of most of the open circular plasmid to supercoiled plasmid.

### EXAMPLE 11

### DNA Ligase+DNA Gyrase

A 10 μl reaction volume contained 5 μg p6 kb plasmid, 35 mM Tris-HCl, pH 7.5, 25 mM KCl, 4 mM MgCl₂, 2 mM dithiothreitol, 1.8 mM spermidine, 1 mM ATP, 6.4% glycerol, 0.1 mg/ml bovine serum albumin, 2.5 units DNA gyrase (E. coli), 2.8 μg GST-T4 DNA ligase. This reaction was incubated at 37 degrees for 2 hours. After the incubation, the plasmid was analyzed by agarose gel electrophoresis. The gel showed conversion of some of the open circular plasmid to supercoiled plasmid.

1. A method for preparing plasmid from host cells which contain the plasmid, comprising the steps:
(a) preparing a cleared lysate of the host cells, wherein the cleared lysate comprises unligatable open circular plasmid;
(b) incubating the unligatable open circular plasmid with one or more enzymes in the presence of their appropriate nucleotide cofactors, whereby the unligatable open circular plasmid is converted to 3′-hydroxyl, 5′-phosphate nicked plasmid;
(c) incubating the 3′-hydroxyl, 5′-phosphate nicked plasmid with DNA ligase in the presence of DNA ligase nucleotide cofactor, whereby 3′-hydroxyl, 5′-phosphate nicked plasmid is converted to relaxed covalently closed circular plasmid; and
(d) incubating the relaxed covalently closed circular plasmid with DNA gyrase in the presence of DNA gyrase nucleotide cofactor, whereby relaxed covalently closed circular plasmid is converted to negatively supercoiled plasmid.

2. A method according to claim 1, wherein the nucleotide cofactor for DNA gyrase is ATP and wherein the step (d) incubation is performed in the presence of a regenerating enzyme and a high energy phosphate donor which convert ADP, generated by DNA gyrase activity, to ATP.

3. A method according to claim 1, wherein the step (c) incubation is performed in the presence of one or more regenerating enzymes and a high energy phosphate donor which convert the nucleotide cofactor by-product of DNA ligase, generated by DNA ligase activity, to nucleotide cofactor.

4. A method according to claim 3, wherein the nucleotide cofactor for DNA ligase is ATP and wherein the step (c) incubation is performed in the presence of one or more regenerating enzymes and a high energy phosphate donor which convert AMP, generated by DNA ligase activity, to ATP.

5. A method according to claim 4, wherein the step (c) incubation is performed in the presence of inorganic pyrophosphatase, whereby pyrophosphate generated by the DNA ligase reaction is converted to phosphate.

6. A method according to claim 1, wherein the cleared lysate further comprises residual linear chromosomal DNA, further comprising the step (e) after step (a) of incubating with one or more exonucleases, wherein said exonuclease enzymes selectively degrade linear chromosomal DNA without degrading open circular plasmid and without degrading relaxed covalently closed circular plasmid and without degrading supercoiled plasmid.

7. A method according to claim 1, wherein the cleared lysate further comprises residual linear chromosomal DNA, further comprising the step (f) after steps (a), (b), and (c) of incubating with one or more exonucleases, wherein said exonuclease enzymes selectively degrade linear chromosomal DNA without degrading relaxed covalently closed circular plasmid and without degrading supercoiled plasmid.

8. A method according to claim 6, wherein one of the exonuclease enzymes is ATP dependent exonuclease.

9. A method according to claim 1, further comprising the step (g) of incubation with DNA topoisomerase IV, whereby plasmid catenanes are decatenated.

10. A method according to claim 1, wherein step (b) is performed by incubating the unligatable open circular plasmid with DNA polymerase I in the presence of deoxyribonucleoside triphosphates.

11. A method according to claim 10, wherein the incubation steps (b), (c), and (d) are combined, by incubating with an enzyme mixture comprising DNA polymerase I, DNA ligase, and DNA gyrase.

12. A method according to claim 11, wherein the enzyme mixture further comprises a regenerating enzyme, wherein said regenerating enzyme converts the nucleotide by-product of DNA gyrase nucleotide cofactor back to nucleotide cofactor in the presence of a high energy phosphate donor.

13. A method according to claim 11, wherein the enzyme mixture further comprises one or more exonucleases, wherein the exonucleases selectively degrade linear chromosomal DNA without degrading open circular plasmid, relaxed covalently closed circular plasmid, and supercoiled plasmid.

14. A method according to claim 1, wherein step (c) is performed without prior purification of 3′-hydroxyl, 5′-phosphate nicked plasmid from step (b) and wherein step (d) is performed without prior purification of covalently closed circular plasmid from step (c).

15. A method according to claim 1, wherein the cleared lysate further comprises supercoiled plasmid and wherein the supercoiled plasmid the open circular plasmid are further purified after step (a) and prior to steps (b), (c), and (d).

16. A method according to claim 1, further comprising the step (f) after steps (a), (b), (c), and (d) of transforming the negatively supercoiled plasmid into recipient cells.

17. A method according to claim 1, wherein the cleared lysate of step (a) is obtained by the steps in sequence:
(i) lysing the host cells which contain the plasmid, thereby releasing plasmid and chromosomal DNA into a lysate solution;
(ii) precipitating the chromosomal DNA from the lysate solution; and
(iii) removing the precipitated chromosomal DNA from the lysate solution, resulting in a cleared lysate.

18. A method according to claim 17, wherein the cells are lysed by using alkaline detergent and wherein the chromosomal DNA is precipitated by neutralizing the lysate solution.

19. A method according to claim 1, wherein the host cell is a bacterium.

20. A method according to claim 1, wherein step (d) incubation is performed in the absence of topoisomerase I.

21. A method according to claim 1, wherein the cleared lysate of step (a) further comprises supercoiled plasmid, and wherein steps (b), (c), and (d) are performed (i) without prior purposeful conversion of the supercoiled plasmid to linear form, and (ii) without prior purposeful conversion of supercoiled plasmid to open circular plasmid, and (iii) without prior purposeful conversion of supercoiled plasmid to relaxed covalently closed circular plasmid, and (iv) without prior purposeful conversion of open circular plasmid of step (a) to single stranded circular DNA.

22. A method for preparing plasmid from host cells which contain the plasmid, comprising the steps:
(a) preparing a cleared lysate of the host cells, wherein the cleared lysate comprises 3′-phosphate, 5′-hydroxyl nicked plasmid;
(b) converting the 3′-phosphate, 5′-hydroxyl nicked plasmid to 3′-hydroxyl, 5′-phosphate nicked plasmid by the steps comprising:
(i) incubation with 3′ phosphatase;
(ii) incubation with polynucleotide kinase;
(c) incubating the 3′-hydroxyl, 5′-phosphate nicked plasmid (b) with DNA ligase in the presence of DNA ligase nucleotide cofactor, whereby 3′-hydroxyl, 5′-phosphate nicked plasmid is converted to relaxed covalently closed circular plasmid; and
(d) incubating the relaxed covalently closed circular plasmid with DNA gyrase in the presence of DNA gyrase nucleotide cofactor, whereby relaxed covalently closed circular plasmid is converted to negatively supercoiled plasmid.

23. A method according to claim 22, wherein the incubation steps (i) and (ii) are combined, by incubating with the enzyme polynucleotide kinase—3′-phosphatase.

24. A method according to claim 22, wherein the incubation steps (b), (c), and (d) are combined, by incubating with an enzyme mixture comprising 3′-phosphatase, polynucleotide kinase, DNA ligase, and DNA gyrase.

25. A method according to claim 24, wherein the enzyme mixture further comprises a regenerating enzyme, wherein said regenerating enzyme converts the nucleotide by-product of DNA gyrase nucleotide cofactor back to nucleotide cofactor in the presence of a high energy phosphate donor.

26. A method according to claim 24, wherein the cleared lysate further comprises linear chromosomal DNA and wherein the enzyme mixture further comprises one or more exonucleases, wherein the exonucleases selectively degrade linear chromosomal DNA without degrading open circular plasmid, covalently closed circular plasmid, and supercoiled plasmid.

27. A method for preparing plasmid from host cells which contain the plasmid, comprising the steps:
(a) preparing a cleared lysate of the host cells, wherein the cleared lysate comprises 3′-blocked open circular plasmid, wherein the 3′-blocked open circular plasmid is not 3′-hydroxyl, 5-phosphate nicked plasmid, and wherein the 3′ terminus of the 3′-blocked open circular plasmid has a blocking group at the 3′ terminus which impairs extension by DNA polymerase.
(b) converting the 3′-blocked open circular plasmid to 3′-hydroxyl, 5′-phosphate nicked plasmid by the steps comprising:
(i) incubation with a 3′ deblocking enzyme; and
(ii) incubation with a DNA polymerase in the presence of deoxyribonucleoside triphosphates;
(c) incubating the 3′-hydroxyl, 5′-phosphate nicked plasmid with DNA ligase in the presence of DNA ligase nucleotide cofactor, whereby 3′-hydroxyl, 5′-phosphate nicked plasmid is converted to relaxed covalently closed circular plasmid; and
(d) incubating the relaxed covalently closed circular plasmid with DNA gyrase in the presence of DNA gyrase nucleotide cofactor, whereby relaxed covalently closed circular plasmid is converted to negatively supercoiled plasmid.

28. A method according to claim 27, wherein the DNA polymerase is DNA polymerase I.

29. A method according to claim 28, wherein the incubation steps (b), (c), and (d) are combined, by incubating with an enzyme mixture comprising 3′-deblocking enzyme, DNA polymerase I, DNA ligase, and DNA gyrase.

30. A method according to claim 29, wherein the enzyme mixture further comprises a regenerating enzyme, wherein said regenerating enzyme converts the nucleotide by-product of DNA gyrase nucleotide cofactor back to nucleotide cofactor in the presence of a high energy phosphate donor.

31. A method according to claim 29, wherein the cleared lysate further comprises linear chromosomal DNA and wherein the enzyme mixture further comprises one or more exonucleases, wherein the exonucleases selectively degrade linear chromosomal DNA without degrading open circular plasmid, covalently closed circular plasmid, and supercoiled plasmid.

32. A method according to claim 27, wherein the 3′-deblocking enzyme is exonuclease III.

33. A method according to claim 27, wherein the 3′-deblocking enzyme is 3′-phosphatase.

34. A method for preparing plasmid from host cells which contain the plasmid, comprising the steps:
(a) preparing a cleared lysate of the host cells, wherein the cleared lysate comprises unligatable open circular plasmid and residual linear chromosomal DNA;
(b) incubating the unligatable open circular plasmid with one or more enzymes in the presence of their appropriate nucleotide cofactors, whereby the unligatable open circular plasmid is converted to 3′-hydroxyl, 5′-phosphate nicked plasmid;
(c) incubating the 3′-hydroxyl, 5′-phosphate nicked plasmid with DNA ligase in the presence of DNA ligase nucleotide cofactor, whereby 3′-hydroxyl, 5′-phosphate nicked plasmid is converted to relaxed covalently closed circular plasmid;
(d) incubating the relaxed covalently closed circular plasmid with DNA gyrase in the presence of DNA gyrase nucleotide cofactor, whereby relaxed covalently closed circular plasmid is converted to negatively supercoiled plasmid; and
(e) incubating with one or more exonucleases, wherein said exonuclease enzymes selectively degrade the linear chromosomal DNA without degrading relaxed covalently closed circular plasmid and without degrading supercoiled plasmid.

35. A method according to claim 34, wherein step (b) is performed by incubating the unligatable open circular plasmid with DNA polymerase I in the presence of deoxyribonucleoside triphosphates.

36. A method according to claim 35, wherein the incubation steps (b), (c), and (d), are combined, by incubating with an enzyme mixture comprising DNA polymerase I, DNA ligase and DNA gyrase.

37. An enzyme composition useful for converting unligatable open circular plasmid to supercoiled plasmid comprising DNA gyrase, DNA ligase, polynucleotide kinase, and 3′-phosphatase.

38. An enzyme composition according to claim 37, further comprising a regenerating enzyme, wherein said regenerating enzyme converts the nucleotide by-product of DNA gyrase nucleotide cofactor back to nucleotide cofactor in the presence of a high energy phosphate donor.

39. An enzyme mixture according to claim 37, further comprising one or more exonucleases, wherein the exonucleases selectively degrade linear chromosomal DNA without degrading open circular plasmid, relaxed covalently closed circular plasmid, and supercoiled plasmid.

40. An enzyme composition useful for converting unligatable open circular plasmid to supercoiled plasmid comprising DNA polymerase I, DNA ligase, and DNA gyrase, and not comprising a primase enzyme.

41. An enzyme composition useful for converting unligatable open circular plasmid to supercoiled plasmid comprising a 3′ deblocking enzyme, DNA polymerase I, DNA ligase, and DNA gyrase.

42. An enzyme composition according to claim 41, further comprising a regenerating enzyme, wherein said regenerating enzyme converts the nucleotide by-product of DNA gyrase nucleotide cofactor back to nucleotide cofactor in the presence of a high energy phosphate donor.

43. An enzyme mixture according to claim 41, further comprising one or more exonucleases, wherein the exonucleases selectively degrade linear chromosomal DNA without degrading open circular plasmid, relaxed covalently closed circular plasmid, and supercoiled plasmid.

44. An enzyme composition according to claim 41, wherein the 3′ deblocking enzyme is exonuclease III.

45. An enzyme composition according to claim 41, wherein the 3′ deblocking enzyme is 3′-phosphatase.

46. An enzyme composition useful for converting unligatable open circular plasmid to supercoiled plasmid comprising DNA polymerase I, DNA ligase, DNA gyrase, and one or more exonucleases, wherein the exonucleases selectively degrade linear chromosomal DNA without degrading open circular plasmid, relaxed covalently closed circular plasmid, and supercoiled plasmid.

47. A method according to claim 1, wherein the steps (b), (c), and (d) are performed without in vitro plasmid replication and without prior in vitro plasmid replication.

48. A method for preparing plasmid from host cells which contain the plasmid, comprising the steps:
(a) preparing a cleared lysate of the host cells;
(b) incubating unligatable open circular plasmid, obtained from the cleared lysate or obtained from supercoiled plasmid from the cleared lysate which is unintentionally converted to unligatable open circular plasmid prior to step (b), with one or more enzymes in the presence of their appropriate nucleotide cofactors, whereby the unligatable open circular plasmid is converted to 3′-hydroxyl, 5′-phosphate nicked plasmid;
(c) incubating the 3′-hydroxyl, 5′-phosphate nicked plasmid with DNA ligase in the presence of DNA ligase nucleotide cofactor, whereby 3′-hydroxyl, 5′-phosphate nicked plasmid is converted to relaxed covalently closed circular plasmid; and
(d) incubating the relaxed covalently closed circular plasmid with DNA gyrase in the presence of DNA gyrase nucleotide cofactor, whereby relaxed covalently closed circular plasmid is converted to negatively supercoiled plasmid.

49. A method according to claim 48, wherein step (b) is performed by incubating the unligatable open circular plasmid with DNA polymerase I in the presence of deoxyribonucleoside triphosphates.

50. A method according to claim 49, wherein the incubation steps (b), (c), and (d) are combined, by incubating with an enzyme mixture comprising DNA polymerase I, DNA ligase, and DNA gyrase.

51. A method according to claim 50, wherein the enzyme mixture further comprises a regenerating enzyme, wherein said regenerating enzyme converts the nucleotide by-product of DNA gyrase nucleotide cofactor back to nucleotide cofactor in the presence of a high energy phosphate donor.

52. A method according to claim 50, wherein the plasmid solution further comprises linear chromosomal DNA, and wherein the enzyme mixture further comprises one or more exonucleases, wherein the exonucleases selectively degrade linear chromosomal DNA without degrading open circular plasmid, relaxed covalently closed circular plasmid, and supercoiled plasmid.

53. A method for preparing plasmid from host cells which contain the plasmid, comprising the steps:
(a) preparing a cleared lysate of the host cells;
(b) converting 3′-phosphate, 5′-hydroxyl nicked plasmid, obtained from the cleared lysate or obtained from supercoiled plasmid from the cleared lysate which is unintentionally converted to 3′-phosphate, 5′-hydroxyl nicked plasmid prior to step (b), to 3′-hydroxyl, 5′-phosphate nicked plasmid by the steps comprising:
(i) incubation with 3′ phosphatase;
(ii) incubation with polynucleotide kinase;
(c) incubating the 3′-hydroxyl, 5′-phosphate nicked plasmid with DNA ligase in the presence of DNA ligase nucleotide cofactor, whereby 3′-hydroxyl, 5′-phosphate nicked plasmid is converted to relaxed covalently closed circular plasmid; and
(d) incubating the relaxed covalently closed circular plasmid with DNA gyrase in the presence of DNA gyrase nucleotide cofactor, whereby relaxed covalently closed circular plasmid is converted to negatively supercoiled plasmid;

54. A method according to claim 53, wherein the incubation steps (i) and (ii) are combined, by incubating with the enzyme polynucleotide kinase—3′-phosphatase.

55. A method according to claim 53, wherein the incubation steps (b), (c), and (d) are combined, by incubating with an enzyme mixture comprising 3′-phosphatase, polynucleotide kinase, DNA ligase, and DNA gyrase.

56. A method according to claim 55, wherein the enzyme mixture further comprises a regenerating enzyme, wherein said regenerating enzyme converts the nucleotide by-product of DNA gyrase nucleotide cofactor back to nucleotide cofactor in the presence of a high energy phosphate donor.

57. A method according to claim 55, wherein the cleared lysate further comprises linear chromosomal DNA and wherein the enzyme mixture further comprises one or more exonucleases, wherein the exonucleases selectively degrade linear chromosomal DNA without degrading open circular plasmid, covalently closed circular plasmid, and supercoiled plasmid.

58. A method for preparing plasmid from host cells which contain the plasmid, comprising the steps:
(a) preparing a cleared lysate of the host cells;
(b) converting 3′-blocked open circular plasmid, obtained from the cleared lysate or obtained from supercoiled plasmid from the cleared lysate which is unintentionally converted to 3′-blocked open circular plasmid prior to step (b), to 3′-hydroxyl, 5′-phosphate nicked plasmid, wherein the 3′-blocked open circular plasmid is not 3′-hydroxyl, 5-phosphate nicked plasmid, and wherein the 3′ terminus of the 3′-blocked open circular plasmid has a blocking group at the 3′ terminus which impairs extension by DNA polymerase, by the steps comprising:
(i) incubation with a 3′ deblocking enzyme; and
(ii) incubation with a DNA polymerase in the presence of deoxyribonucleoside triphosphates;
(c) incubating the 3′-hydroxyl, 5′-phosphate nicked plasmid with DNA ligase in the presence of DNA ligase nucleotide cofactor, whereby 3′-hydroxyl, 5′-phosphate nicked plasmid is converted to relaxed covalently closed circular plasmid; and
(d) incubating the relaxed covalently closed circular plasmid with DNA gyrase in the presence of DNA gyrase nucleotide cofactor, whereby relaxed covalently closed circular plasmid is converted to negatively supercoiled plasmid;

59. A method according to claim 58, wherein the DNA polymerase is DNA polymerase I.

60. A method according to claim 59, wherein the incubation steps (b), (c), and (d) are combined, by incubating with an enzyme mixture comprising 3′-deblocking enzyme, DNA polymerase I, DNA ligase, and DNA gyrase.

61. A method according to claim 60, wherein the enzyme mixture further comprises a regenerating enzyme, wherein said regenerating enzyme converts the nucleotide by-product of DNA gyrase nucleotide cofactor back to nucleotide cofactor in the presence of a high energy phosphate donor.

62. A method according to claim 60, wherein the cleared lysate further comprises linear chromosomal DNA and wherein the enzyme mixture further comprises one or more exonucleases, wherein the exonucleases selectively degrade linear chromosomal DNA without degrading open circular plasmid, covalently closed circular plasmid, and supercoiled plasmid.

63. A method according to claim 58, wherein the 3′-deblocking enzyme is exonuclease III.

64. A method according to claim 58, wherein the 3′-deblocking enzyme is 3′-phosphatase.

65. A method for preparing plasmid from host cells which contain the plasmid, comprising the steps:
(a) preparing a cleared lysate of the host cells, wherein the cleared lysate comprises residual linear chromosomal DNA;
(b) incubating unligatable open circular plasmid, obtained from the cleared lysate or obtained from supercoiled plasmid from the cleared lysate which is unintentionally converted to unligatable open circular plasmid prior to step (b), with one or more enzymes in the presence of their appropriate nucleotide cofactors, whereby the unligatable open circular plasmid is converted to 3′-hydroxyl, 5′-phosphate nicked plasmid;
(c) incubating the 3′-hydroxyl, 5′-phosphate nicked plasmid with DNA ligase in the presence of DNA ligase nucleotide cofactor, whereby 3′-hydroxyl, 5′-phosphate nicked plasmid is converted to relaxed covalently closed circular plasmid;
(d) incubating the relaxed covalently closed circular plasmid with DNA gyrase in the presence of DNA gyrase nucleotide cofactor, whereby relaxed covalently closed circular plasmid is converted to negatively supercoiled plasmid; and
(e) incubating with one or more exonucleases, wherein said exonuclease enzymes selectively degrade the linear chromosomal DNA without degrading relaxed covalently closed circular plasmid and without degrading supercoiled plasmid.

66. A method according to claim 65, wherein step (b) is performed by incubating the unligatable open circular plasmid with DNA polymerase I in the presence of deoxyribonucleoside triphosphates.

67. A method according to claim 66, wherein the incubation steps (b), (c), and (d) are combined, by incubating with an enzyme mixture comprising DNA polymerase I, DNA ligase, and DNA gyrase.

68. A method for preparing plasmid from host cells which contain the plasmid, comprising the steps:
(a) preparing a cleared lysate of the host cells; and
(b) in vitro enzymatically converting open circular plasmid to supercoiled plasmid, wherein the open circular plasmid is obtained from the cleared lysate or obtained from supercoiled plasmid from the cleared lysate which is unintentionally converted to open circular plasmid prior to step (b).

69. A method according to claim 68, wherein the open circular plasmid comprises 3′-hydroxyl, 5′-phosphate nicked plasmid, and wherein the 3′-hydroxyl, 5′-phosphate nicked plasmid is converted to supercoiled plasmid by the steps comprising:
(i) incubating the 3′-hydroxyl, 5′-phosphate nicked plasmid with DNA ligase in the presence of DNA ligase nucleotide cofactor, whereby 3′-hydroxyl, 5′-phosphate nicked plasmid is converted to relaxed covalently closed circular plasmid; and
(ii) incubating the relaxed covalently closed circular plasmid with DNA gyrase in the presence of DNA gyrase nucleotide cofactor, whereby relaxed covalently closed circular plasmid is converted to negatively supercoiled plasmid.

70. A method according to claim 69, wherein the cleared lysate further comprises linear chromosomal DNA, further comprising the step (c) of incubating with one or more exonucleases, wherein said exonucleases selectively degrade linear chromosomal DNA without degrading supercoiled plasmid.

71. A method for preparing highly supercoiled plasmid from host cells which contain host supercoiled plasmid, comprising the steps:
(a) preparing a cleared lysate of the host cells, wherein the cleared lysate comprises the host supercoiled plasmid;
(b) enzymatically in vitro converting open circular plasmid to supercoiled plasmid, wherein the open circular plasmid is obtained from the cleared lysate or obtained from supercoiled plasmid from the cleared lysate which is unintentionally converted to open circular plasmid prior to step (b); and
(c) incubating in vitro the host supercoiled plasmid with DNA gyrase in the presence of DNA gyrase nucleotide cofactor, whereby the host supercoiled plasmid is further supercoiled;

72. A method according to claim 71, wherein the open circular plasmid comprises 3′-hydroxyl, 5′-phosphate nicked plasmid, and wherein the 3′-hydroxyl, 5′-phosphate nicked plasmid is converted to supercoiled plasmid by the steps comprising:
(i) incubating the 3′-hydroxyl, 5′-phosphate nicked plasmid with DNA ligase in the presence of DNA ligase nucleotide cofactor, whereby 3′-hydroxyl, 5′-phosphate nicked plasmid is converted to relaxed covalently closed circular plasmid; and
(ii) incubating the relaxed covalently closed circular plasmid with DNA gyrase in the presence of DNA gyrase nucleotide cofactor, whereby relaxed covalently closed circular plasmid is converted to negatively supercoiled plasmid.

73. A method according to claim 71, wherein the open circular plasmid comprises unligatagable open circular plasmid, and wherein the unligatable open circular plasmid is converted to supercoiled plasmid by the steps comprising:
(i) incubating the unligatable open circular plasmid with one or more enzymes in the presence of their appropriate nucleotide cofactors, whereby the unligatable open circular plasmid is converted to 3′-hydroxyl, 5′-phosphate nicked plasmid;
(ii) incubating the 3′-hydroxyl, 5′-phosphate nicked plasmid with DNA ligase in the presence of DNA ligase nucleotide cofactor, whereby 3′-hydroxyl, 5′-phosphate nicked plasmid is converted to relaxed covalently closed circular plasmid; and
(iii) incubating the relaxed covalently closed circular plasmid with DNA gyrase in the presence of DNA gyrase nucleotide cofactor, whereby relaxed covalently closed circular plasmid nverted to negatively supercoiled plasmid.
